(19) United States
(12) Patent Application Publication
(10) Pub. No.: US 2002/0123790 A1
(43) Pub. Date: Sep. 5, 2002

(54) Enhanced engagement member for anchoring prosthetic devices in body lumen

(76) Inventors: Geoffrey Hamilton White, Birchgrove (AU); Mark Dehdashtian, Costa Mesa, CA (US)

Correspondence Address: PILLSBURY WINTHROP LLP, Suite 2800, 725 South Figueroa Street, Los Angeles, CA, 90017, US

(21) Appl. No.: 09/966,567

(22) Filed: Sep. 27, 2001

(30) Foreign Application Priority Data
Sep. 28, 1999 (EP) 99950401.2

(51) Int. Cl. A61F 2/06

(52) U.S. Cl. 623/1.14; 623/1.36; 623/1.35; 623/1.31

(57) ABSTRACT

Novel intraluminal devices that include a plurality of engagement members to minimize graft migration and vessel wall damage are described. Applications of the instant teachings for AAA and TAA issues wherein proximal neck attachments are critical are featured, in addition to general usages with stenotic disease. Further, an enhanced engagement member is described for preventing the migration of a prosthetic device through a body lumen.

### RELATED APPLICATIONS

This application relates generally to U.S. patent application Ser. No. 09/806,179 filed Mar. 28, 2001, now abandoned, which claims full Paris Convention Priority from PCT Application AU99/00832, filed Sep. 28, 1999, and to European Patent Application No. 99950401.2, filed Sep. 28, 1999, both of which are incorporated herein by reference in their entirety.

### FIELD OF THE INVENTION

The present invention relates to an intraluminal device for use in the treatment of aneurysmal or stenotic disease, and methods of emplacing and fixing such devices within the human body.

### BACKGROUND OF THE INVENTION

Since the early 1990's stented tubular members made of various materials (“stent-grafts”) have become known within the context of treating aortic and peripheral vascular disease without the need for conventional open surgery, which carries a high mortality rate.

It is known to use intraluminal grafts and stents of various designs for the treatment of aneurysms such as aortic aneurysms, and occlusive diseases affecting the vasculature or other vessels comprising, inter alia, the hepato-biliary and genito-urinary tracts (hereinafter “vessels”). It is known to form such an intraluminal device of a sleeve in which is disposed a plurality of self-expanding wire stems (see Balko A. et al. (1986) Transfemoral Placement of Intraluminal Polyurethane Prosthesis for Abdominal Aortic Aneurysms Journal of Surgical Research 40, 305-309; Mirich D. et al. (1989) Percutaneously Placed Endovascular Grafts for Aortic Aneurysms: Feasibility Study Radiology 170(3), 1033-1037).

In the past, such devices have commonly been used in the treatment of aneurysms. However it has been recognized that it is within the ambit of some such devices that they also be used to treat stenotic lesions. Whichever the purpose for which an intraluminal device is being used, it has the capacity to be inserted percutaneously through a connecting vessel either distal or proximal to that in which the device is to be used. For example, it may be inserted through the femoral artery in a catheter, where the device is used in the treatment of a lesion within the aorta. Upon release of the device from the catheter it may expand or be expanded to a desirable size. Furthermore, it may be placed to extend both above and below the lesion, thereby bridging the lesion. This method of inserting the device into the body of a patient is applicable where the invention is used in the treatment of aneurysmal disease or stenotic disease.

There are a number of problems associated with such known intraluminal devices. First, there is the problem of maintaining the device against longitudinal movement, or migration, along the lumen in which it is placed. Secondly, there is no known means to engage other instruments or devices to the intraluminal device should that be desirable at any stage throughout the life of the device. Likewise, the need for both long-term compliance of stent-grafts and sustained durability for periods of greater than ten years have presented new engineering and design obstacles.

Although the first of these problems has been addressed by prior inventions (see, for example: PCT/AU94/00586 entitled “Intraluminal Graft” in the name of Endogad Research Pty Limited, issued to common assignee Edwards Lifesciences Corporation as U.S. Pat. No. 5,782,904, and U.S. Pat, No. 5,282,824 entitled “Percutaneous Stent Assembly” in the name of Gianturco), the present inventor has found that there are alternative means to overcome that problem, and others, and has incorporated them into this invention.

Thus, the present invention is directed to an improved anti-migration aspect of intraluminal devices which serves to ameliorate the above problems as well as provide yet other benefits. The devices that are within the range of the present invention are effective as bifurcated endoluminal prostheses, or may alternatively be used, for example, as “cuffs” or “extensions” as disclosed in commonly assigned U.S. application Ser. Nos. 09/473,618 (filed Dec. 29, 1999); 09/478,413 (filed Jan. 6, 2000); and 09/478,352 (filed Jan. 6, 2000) which are expressly incorporated herein by reference in their entirety. Likewise, the following of applicants' commonly assigned U.S. Patents are expressly incorporated by reference as if fully set forth herein:

| | | |
|---|---|---|
| 5,968,068 | 6,013,092 | 6,024,729 |
| 6,071,307 | 6,045,557 | 6,099,558 |
| 6,106,540 | 6,110,191 | 6,143,014. |

### SUMMARY OF THE DISCLOSURE

The present invention consists of an intraluminal device comprising a tubular body having at least two ends which is capable of expanding or being expanded from a radially compressed state to a radially expanded state, and at least a plurality of engagement members being connected to or integral with a wall of the device body at a position located between the ends of the device body, the connection between the engagement member and the device body being such that the engagement member may occupy a first angular relationship with an adjoining part of the device body when the device body is radially compressed and a second and different angular relationship with an adjoining part of the device body when the device body is radially expanded. The engagement members, or “crimps,” engage the wall of a vessel lumen to inhibit longitudinal movement of the device therein.

In another aspect, the invention consists of an intraluminal device comprising a tubular body with two ends which is capable of expanding or being expanded from a radially compressed state to a radially expanded state in vivo in the body of a patient, and at least one engagement member which is connected to or integral with a wall of the device body, the connection between and materials of the engagement member and the device body being such that the engagement member may occupy a first angular relationship with an adjoining part of the device body when the device body is radially compressed and a second and different angular relationship with an adjoining part of the device body when the device body is radially expanded in vivo in the body of a patient. The wireform crimps preferably include sleeves over their tail segments and wire ends extending past the sleeves, directly engaging inside surfaces of body lumens.

Still another aspect of the invention relates to a method for positioning an intraluminal device according to other enumerated aspects of the invention. The method involves the steps of: introducing a catheter or other delivery device into a vein, artery or other vessel in the body of a patient when the device body is in a radially compressed state and the engagement member occupies a first angular relationship with the device body; causing the intraluminal device to be carried through the catheter or other delivery device until the intraluminal device extends into the vessel from the catheter or other delivery device; causing or allowing the intraluminal device to expand; causing or allowing the engagement member to occupy its second angular relationship with the device body; and withdrawing the catheter or other deliver device along with any other apparatus used to introduce the intraluminal device into the vessel.

The intraluminal device according to this invention may be used in the treatment of aneurysms or stenotic disease. In addition to treating aortic aneurysms, the device is particularly suitable for treating aneurysms of the femoral artery, the popliteal artery, the thoracic segment of the aorta, visceral arteries such as the renal and mesenteric arteries, the iliac artery and the sub-clavian artery. Further, in addition to the treatment of stenotic lesions in the peripheral vasculature, the invention may be used in the treatment of, inter alia, vessels comprising the coronary circulation. The application of the invention for use in the treatment of stenotic disease is not to be understood as limited to the vascular system only, the device may be used to treat stenotic lesions in other vessels including, for example, those comprising the hepato-biliary and genito-urinary tracts.

In cases where the invention is to be used for the treatment of aneurysmal disease, the tubular device body is preferably formed of a thin biocompatible material such as DACRON (E.I. DuPont de Nemours & Company, Delaware, hereinafter “Dacron”) or expanded poly-tetrafluoroethylene (hereinafter “PTFE”), and/or combinations thereof. The device body may further take the form of bifurcated endoluminal prostheses, extensions, cuffs and like devices for customizing a stent-graft system to a patient's anatomy. Any involved tube material may be preferably crimped along its length to increase the device's flexibility; however, uncrimped material may be used in suitable circumstances. In preferred embodiments of the invention for use in the treatment of aneurysmal disease, the device body may be formed from a material having a limited amount of diametric elasticity to ensure that it can be expanded into contact with the vessel wall, forming a seal between the wall of the device body and the vessel wall such that escape of the vessel contents into the aneurysmal sac is prevented.

In addition, some embodiments of the invention for use in the treatment of aneurysmal disease may be such that the device body includes a stent or a series of spaced apart stents that form a framework to which an endoluminal graft may be attached. The framework may be a plurality of separate, spaced-apart, malleable wires. Each of such wires may have a generally closed sinusoidal or zig-zag shape, also referred to as an “undulating” shape. The wires are preferably formed of stainless steel or another metal or plastic that is both malleable and biocompatible. Each wire is preferably woven into the fabric of the device body to integrate the body and the reinforcing wires. This minimizes the possibility of the wire reinforcement separating from the device body either during introduction of the device or at a later time. Where a woven material is utilized to form the device body, the wires may be interwoven with the device body. This is preferably accomplished after the device body is manufactured. If the device body is not constructed of a woven material but rather is knitted or made of an impervious sheet material, then the wires may be threaded through suitable holes provided in the device body. Alternatively, the stent or stents may be continuous and may be on the radially inner or the radially outer surface of the device wall. In either case, expansion of the stent or stents will cause the graft to expand and press against the wall of a vessel into which the device has been placed.

In yet further embodiments of the invention for use in the treatment of aneurysmal disease, the wires described above may be held in place by sutures or adhesives or may be sandwiched between layers of a multi-layered tubular device body. In each of the foregoing embodiments, the wires are preferably disposed substantially within the device body. It is within the ambit of the invention, however, that the wires may alternatively be connected to, and disposed upon, the outer surface of the device body.

Likewise, supra renal fixation structures, including self-expanding barred wireforms and radiographic markers, tapers, and the teachings of applicants' expressly incorporated patents and serial numbers are contemplated as integral to the teachings of the present invention.

In cases where the present invention is to be used for the treatment of stenotic disease, the tubular device body is preferably formed of a thin biocompatible material such as Nitinol (nickel titanium alloy, hereinafter “Nitinol”), stainless steel, tantalum, or ELGILOY (American Gage & Machine Company, Illinois, hereinafter “Elgiloy”) (a cobalt-chromium-nickel alloy). The device body may be bare or may be coated with material having an elastic property such that the coating material covers the device body in both its radially compressed and radially expanded states. In preferred embodiments of the present invention for use in the treatment of stenotic disease, the device body may be formed from other suitable biocompatible materials, selected, for best results, on the basis of the material's capacity to withstand the compressive forces of the stenotic lesion and maintain patency of the vessel throughout the life of the device.

In all the foregoing embodiments of the invention, including those for use of the invention in the treatment of aneurysmal disease and those for use of the invention for the treatment of stenotic disease, the length and radially expanded diameter of the device body may be determined by the individual circumstances of the application to which the intraluminal device is to be put. Typically, the vessel will be assessed by X-ray or similar diagnostic technique and a suitably dimensioned device will be selected for that application.

The ability of the device body to change or to be changed from a radially compressed state to a radially expanded state is an important feature of the present invention. It is desirable, for the purpose of introducing the device into the selected vessel, that the device occupies the smallest possible radial diameter along its length. Thus, in a preferred embodiment, the device body will initially be radially compressed. Once the invention has been deployed into the selected vessel and positioned appropriately, the device body may be caused to expand, or may be allowed to self-expand.

There are at least three preferred mechanisms whereby the device body may change from a radially compressed state to a radially expanded state. These are: (1) expansion effected by the physical force of an inflating balloon within the device body or by some other mechanically applied force (“mechanical expansion”); (2) self-expansion following the introduction of the device body into the body of a patient, wherein a patient's body temperature causes the temperature of the device body to rise, thereby causing the device body to self-expand (“thermal expansion”); and, (3) self-expansion following deployment of the invention from the catheter used to introduce the invention into the body of the patient, wherein a property of the material comprising the device body is a “memory” of a preferred shape of the device body in the radially expanded state, such that the device body may “spring” into that state upon release from the catheter (“spring expansion”). Any combinations of the above-mentioned mechanisms are preferred in accordance with the teachings of the present invention.

In cases where “mechanical expansion” is the method for causing the device body to change from a radially compressed state to a radially expanded state, a surgeon's intervention will be required to cause that change. Following introduction of a catheter into a selected vessel in the body of a patient, the device may be caused to be carried through the catheter on an inflatable balloon until the device extends into the vessel from the proximal end of the catheter. Once the preferred position for the device is achieved, the balloon may be inflated such that it causes the device body to expand and therefore acquire a radially expanded state. The method of “mechanical expansion” is one in which the surgeon may maintain the rate at which, and extent to which, the device body expands. In preferred embodiments of the invention, wherein the selected method for changing the device body from a radially compressed state to a radially expanded state is “mechanical expansion,” materials such as Dacron or PTFE are particularly amenable for use in the manufacture of the wall of the device body. As an alternative to the use of a balloon to cause mechanical expansion, other mechanical means, such as a screw jack, may be used to effect expansion of the device body.

Where “thermal expansion” is the method for causing the device body to change from a radially compressed state to a radially expanded state, such change does not require specific intervention by the surgeon. In this case the device body, upon being introduced into the body of a patient, undergoes an increase in temperature caused by its placement within the body of the patient. Consequently, the device will change its shape such that it achieves a radially expanded state. In embodiments of the invention where “thermal expansion” is employed, it may be necessary, before using the invention, to predetermine the desired radially expanded diameter of the device body so that the radial size of the device is appropriate to the circumstances of the particular case. In preferred embodiments of the invention, wherein “thermal expansion” of the device body is employed, materials such as Nitinol are preferably used in the manufacture of the device body.

In cases where “spring expansion” is the method for causing the device body to change from a radially compressed state to a radially expanded sate, the method for positioning the device is a defining feature of the invention. In preferred embodiments of the invention where “spring expansion” is employed, the device body may be manufactured from a material, such as stainless steel wire, which has the capacity to “memorize” its manufactured shape, such that it has a continuous tendency to return to that shape following any events which cause it to be temporarily deformed. Thus a device where “spring expansion” is the preferred method for causing the device body to change from a radially compressed state to a radially expanded state will typically be manufactured such that it is initially in a radially expanded state. In preferred embodiments of the invention where “spring expansion” is used, the method for positioning such a device involves: introducing a catheter into a selected vessel; manually compressing the device body into a radially compressed state; inserting the device with its body maintained in the radially compressed state into the catheter; causing the device to be carried through the catheter until the device extends into the vessel from the proximal end of the catheter (or from some other part of the catheter or other delivery device as may be used to introduce the intraluminal device into the vessel), thereby enabling the device body to “spring” back into a radially expanded state (having been released from the confines of the catheter lumen or any other instrument which had been maintaining the device body in a radially compressed state); and withdrawing the catheter along with any other apparatus used to introduce the device into the vessel.

The methods described above for causing the expansion of the device body from a radially compressed state to a radially expanded state are by no means representative of an exhaustive list. Many alternative methods including, but not limited to, the use of electromagnetic fields and electric currents are well within the scope of the invention.

Engagement members are also utilized in various embodiments of the present invention. The engagement members prevent the device from migrating or moving longitudinally within the vessel following deployment of the device. In preferred embodiments of the invention there are a plurality of engagement members connected to or integral within the wall of the device body.

Preferred embodiments of the present invention are not designed to pierce vessel walls, or impact harmfully upon the intima of lumens. Rather, they serve to maximize the stability and, for example, proximal neck fixation when used for AAA and TAA applications.

In embodiments of the invention wherein the engagement members are connected to the wall of the device body, such connection is preferably created during the manufacture of the device. The elected method for achieving such connection will primarily depend on the material selected to comprise the engagement members and that selected to comprise the device body. The scope of this invention does include, however, all combinations of the selected material for the engagement members and the device body, including the combination in which the material selected for each of these respective components of the invention is the same. Thus, while one means of creating the connection between engagement members and device body may be appropriate for one particular combination of selected materials, an entirely different means may be more appropriate for one of the other possible combinations of selected materials for the respective components of the invention.

In embodiments of the invention wherein the engagement members are integral with a wall of the device body, the engagement members may be formed of the same material as that of the device body. In such cases, the construction of the engagement members will depend upon the construction of the device body. For example, the device body may be formed such that it is circumferentially reinforced along its length by a plurality of separate, spaced-apart, malleable wires, each of which has a generally closed sinusoidal or zig-zag shape. In such an embodiment, the construction of the engagement members may be such that they extend from any one, a plurality, or all of the peaks and/or troughs comprising the sinusoidal or zig-zag shape of those wires.

According to an embodiment of the present invention, there is provided a novel enhanced, specifically configured engagement member for anchoring prosthetic devices within lumens of vessels comprising at least about a plurality of integral barbed extension projections, said at least about a plurality of integral barbed extension projections disposed in a fixed geometric pattern upon an outer surface of an endoluminal graft. In preferred embodiments, the barbed extension projections stabilize the endoluminal graft at a particular location in a vessel by engaging the surrounding vessel tissue without puncturing the same. The extension projections extend out from the endoluminal graft at a relatively small angle, so as to minimize the possibility of such puncturing. Unlike known anchoring means, no penetration of interior aspects of lumens is taught in accordance with the present invention.

In all foregoing embodiments with respect to the engagement members of the invention, the engagement members are preferably formed of a biocompatible material such as Nitinol, stainless steel, Elgiloy, or the like. However, other material may also be appropriate for use in the manufacture of the engagement members including plastic materials that may be resorbable. The engagement members may be coated with materials that promote adhesion of cells or cell growth to assist in securing the device body in place in the vessel. In addition, although in certain circumstances it may be preferable for all of the engagement members to be either connected to or integral with a wall of the device body, the invention may also be such that only a proportion of the engagement members are connected to a wall of the device body, while the remainder are integral with the same or another wall of the device body. However, the scope of the invention does not limit in any way, the number of possible arrangements by which some of the engagement members are connected to a wall of the device body and others are integral with that or another wall of the device body.

The engagement members may be of differing lengths and may be positioned at different locations on the wall of the device body. In addition to occupying different first and second angular relationships with respective adjoining parts of the device body pre- and post-deployment in a vessel, each engagement member may further occupy different first and second angular relationships with respect to one another. However, the scope of this invention also includes an embodiment wherein the engagement members maintain similar angular relationships with respect to one another, in either their first or second angular positions, or both. In one such embodiment of the invention, a group of engagement members may be positioned such that they are spaced apart, surrounding a wall of the device body in the same circumferential plane. When that group of engagement members change from their first angular relationship to occupying a second angular relationship, together they form a “skirt-shaped” extension of a wall of the device body. Such an arrangement of engagement members may be desirable in certain circumstances.

In preferred embodiments of the invention, the relationship between the device body and the engagement members is such that when the device body is in a radially compressed state, the respective first angular relationships of the engagement members may be either flat, running along, or forming a part of the wall of the device body or, alternatively, the engagement members may project inwardly, within the lumen of the device body. Such first angular relationships of the engagement members is of considerable value in ensuring that the smallest possible diameter along the length of the device body is maintained when the device body is in a radially compressed state. For reasons previously explained, it is most desirable, for the purpose of introducing the device into the selected vessel, that the device occupies a small radial diameter along its length.

Once the invention has been introduced into the selected vessel and positioned appropriately, the engagement members may be caused to change from occupying their first angular relationship to occupying respective second angular relationships, or the engagement members may make such a change without specific assistance from the surgeon.

There are at least four preferred mechanisms whereby the engagement members may change from having a first angular relationship with an adjoining part of a wall of the device body to having a second angular relationship with an adjoining part of the wall of the device body. These are: (1) change of angular relationship effected by the physical force of an inflating balloon or other mechanical device (“mechanically-aided change”); (2) self-change following the introduction of the invention into the body of a patient, wherein a patient's body temperature causes the temperature of the engagement members to rise, thereby enabling the engagement members to change from their first angular relationship to their second angular relationship (“heat-aided change”); (3) self-change following deployment of the invention from the catheter or similar device used to introduce the invention into the body of a patient, wherein a property of the material comprising the engagement members is a “memory” of a preferred second angular relationship position, such that the engagement members may “spring” into that position upon release from the catheter or similar device (“spring-aided change”); and, (4) change of angular relationship effected by the change in the geometry of the device body as it expands from a radially compressed state to a radially expanded state (“geometry-aided change”).

In cases where “mechanically-aided change” is the method for causing the engagement members to change from their first angular relationship to a second angular relationship, a balloon may be used to cause such change. Such balloon may therefore be specifically preshaped to suit the particular device with which it will be used. Alternatively, however, the balloon to be used may not require any specific manufacturing arrangements that are out of the ordinary. Where the balloon is preshaped, it may be manufactured such that when inflated it has a series of dimples, between each of which the surface of the balloon does not bulge out as far as it does where the dimples are located. The dimples may be strategically located such that they will push respective engagement members into their second angular relationship when the device is introduced into the body of a patient.

Regardless of whether or not the balloon is specifically preshaped, the “mechanically-aided change” procedure is effectively the same. Once the preferred position for the device is achieved, and the device body has been expanded from a radially compressed state to a radially expanded state in accordance with any of the methods described above, the balloon may be inflated such that its outer surface comes into contact with and presses against the inner surface of the device body. As the balloon continues to inflate, the increasing pressure causes the engagement members to move into their respective second angular relationship positions.

The method of “mechanically-aided change” is one in which the surgeon may maintain the rate at which, and extent to which, the engagement members will change from a first angular relationship to a second angular relationship. It should be noted that in embodiments of the invention, wherein the selected method for causing the change in angular relationships of the engagement members is “mechanically-aided change”, materials such as titanium wire are particularly amenable for use in the manufacture of the invention.

As noted above, the process of “mechanically-aided change” may be induced by a screw jack or other mechanical means that is introduced through the catheter or other delivery device along with the intraluminal device.

Where “heat-aided change” is the method for causing the engagement members to change from a first angular relationship to a second angular relationship, such change does not require specific intervention by the surgeon. In that case, the engagement members, upon the invention being introduced into the body of a patient, will undergo an increase in temperature caused by placement within the body of the patient, and will consequently change their angular relationship such that they acquire a second angular relationship with an adjoining part of the device body as compared to the first. In embodiments of the invention wherein “heat-aided change” is employed, materials such as Nitinol are preferably used in the manufacture of the engagement members.

In alternate embodiments of the present invention, wherein “heat-aided change” is used to change the angular relationships of the engagement members, it is possible to infuse the device with a heated liquid just prior to, or after, placement of the device in the vessel. This obviates the need to rely on body heat alone to induce “heat-aided change” of the engagement members. For instance, the device may be placed in a vessel yet the change in the relative position of the engagement members may be actuated at a later time. Thus, if the device showed signs of moving in the vessel, it may be secured in position by infusing into the vessel a liquid at a temperature above body temperature sufficient to cause the engagement members to change into their second relative positions.

Where “spring-aided change” is the method for causing the angular relationship of the engagement members to change, particular methods for positioning the device may be employed. In preferred embodiments of the invention where “spring-aided change” is employed, the engagement members are manufactured from a material, such as stainless steel, which has the ability to “memorize” its manufactured shape. Such materials have a tendency to return to their manufactured shape following events that cause the material to be temporarily deformed. Thus, a device using “spring-aided change” to cause the engagement members to change from a first angular relationship to a second angular relationship will be manufactured such that the engagement members are initially in the second angular relationship position. In preferred embodiments of the invention where “spring-aided change” is used, the method for positioning such a device involves: introducing a catheter into a selected vessel; manually compressing the engagement members into their first angular relationship positions such that in combination with the device body the invention has the smallest possible radial diameter along its length; inserting the device with the engagement members maintained in their first angular relationship positions into the catheter; causing the device to be carried through the catheter until the device extends into the vessel from the proximal end of the catheter, thereby enabling the engagement members to “spring” back into their respective second angular relationship positions (having been released from the confines of the catheter lumen or other similar instrument which had been maintaining the engagement members in their first angular relationship positions); and withdrawing the catheter along with any other apparatus used to introduce the device into the vessel.

Where “geometry-aided change” is the method for causing the engagement members to change from a first angular relationship position to a second angular relationship position, the construction of the invention is of particular relevance. In such embodiments, there is a physical relationship between the expansion of the device body (from a radially compressed state to a radially expanded state) and the change in angular relationship of the engagement members (from a first to a second angular relationship). That is, the change in the geometry of the device body as it expands causes the engagement members to change from their first angular relationship to a second angular relationship.

As is the case with the expansion of the device body from a radially compressed state to a radially expanded state, the methods described above for causing the engagement members to change from a first angular relationship to a second angular relationship are by no means representative of an exhaustive list. Many alternative methods, including the use of electromagnetic fields and electric currents are within the scope of the present invention.

Having occupied their respective second angular relationships, the engagement members will come into contact with the vessel wall and by so doing, provide resistance to any tendency for longitudinal migration of the device body through the lumen. Since perforation of the lumen is least desirable, the device of the present invention most preferably only engages the wall without causing substantial tissue damage.

In embodiments of the invention wherein at least one of the engagement members remains within the lumen of the device body once in its second angular position, the engagement member may act as a means for engaging other instruments or devices at any state throughout the life of the invention.

### BRIEF DESCRIPTION OF THE DRAWINGS

Hereinafter is an explanation of preferred embodiments of the present invention described with reference to the accompanying diagrams:

FIG. 1 is a partially cut-away ventral view of a patient with an aortic aneurysm that has been bridged by an intraluminal device according to an embodiment of the present invention.

FIG. 2 is a longitudinal sectional view of a vessel with a stenotic lesion and a device according to an embodiment of the present invention with the device body in a radially compressed state in the lumen of the vessel.

FIG. 3a is a longitudinal view of the device with its body in a radially compressed state and engagement members in a respective first angular relationship in accordance with an embodiment of the present invention.

FIG. 3b is a longitudinal view of the device with its body in a radially expanded state and engagement members in a respective second angular relationship in accordance with an embodiment of the present invention.

FIG. 4a is a perspective view of a circumferential section of the device in accordance with an embodiment of the present invention illustrating the engagement members connected to a wall of the device body.

FIG. 4b is a perspective view of a circumferential section of the device in accordance with an embodiment of the present invention illustrating the engagement members integrated within a wall of the device body.

FIG. 5 depicts a preferred embodiment of the device in accordance with the present invention, wherein engagement members are dispersed upon the exterior of a bifurcated prosthesis.

FIG. 6a is a longitudinal view of a device with its body in a radially compressed state, and engagement members integrated within the wall of the device body in a respective first angular relationship in accordance with an embodiment of the present invention.

FIG. 6b is a longitudinal view of a device with its body in a radially expanded state, and engagement members integrated within the wall of the device body, splayed out the from the wall of the device body in a respective second angular relationship in accordance with an embodiment of the present invention.

FIG. 7 is a longitudinal view of a device in accordance with an alternate embodiment of the present invention.

FIG. 8 shows a preferred embodiment of the device in accordance with the present invention wherein engagement members work in complement with a supra renal wireform, bifurcated prosthesis, or aorto-uniliac stent-graft.

FIG. 9 is a side view of an engagement member shown at Y in FIG. 8 in accordance with a preferred embodiment of the present invention.

FIG. 10 is a top view of an engagement member shown at Y in FIG. 8 in accordance with an embodiment of the present invention.

### DETAILED DESCRIPTION OF THE PREFERRED EMBODIMENTS

The present invention is directed toward a novel enhanced intraluminal device including a plurality of engagement members to minimize graft migration without damaging vessel walls. Such vessels may include, but are not limited to, blood vessels as well as the hepato-biliary and genito-urinary tracts. Applications of the instant teachings for AAA and TAA issues wherein proximal neck attachments are critical are featured, in addition to general usages with stenotic disease.

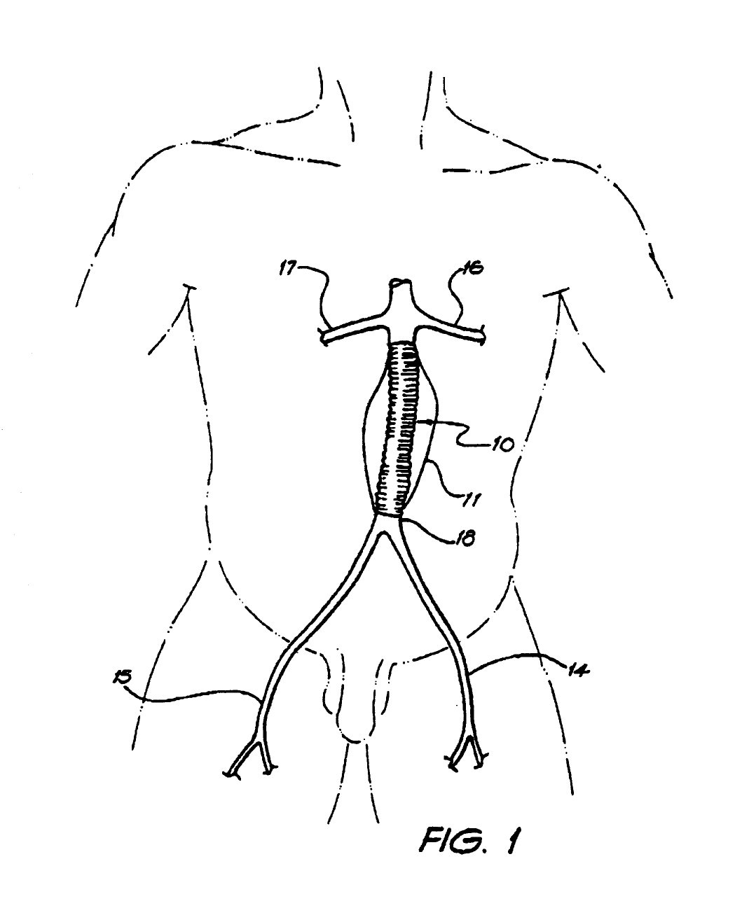

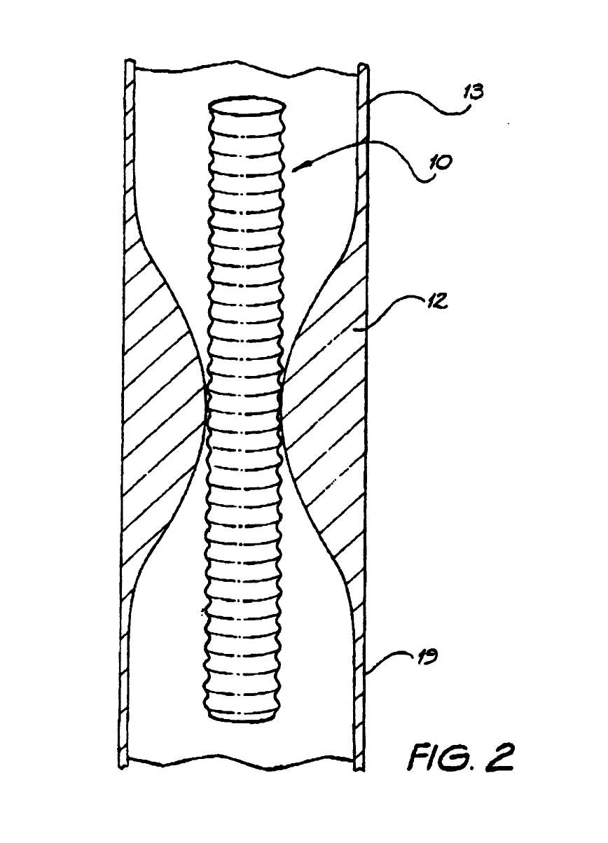

As depicted in FIGS. 1 and 2, in different embodiments, the device 10 may be used in the treatment of aneurysmal disease, such as an aneurysm of the aorta 11 or in the treatment of a stenotic lesion 12 within a vessel 13.

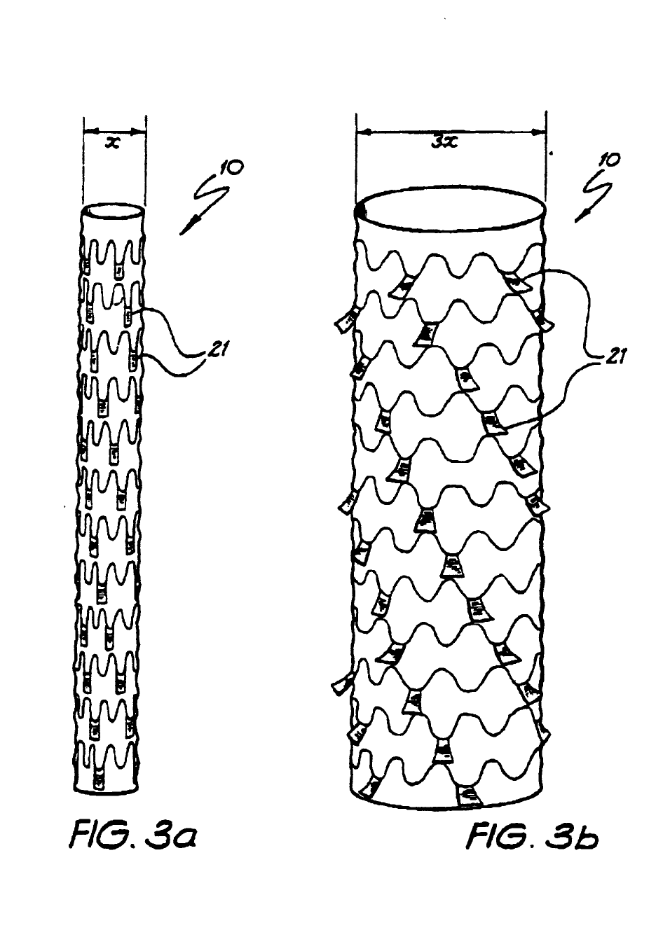

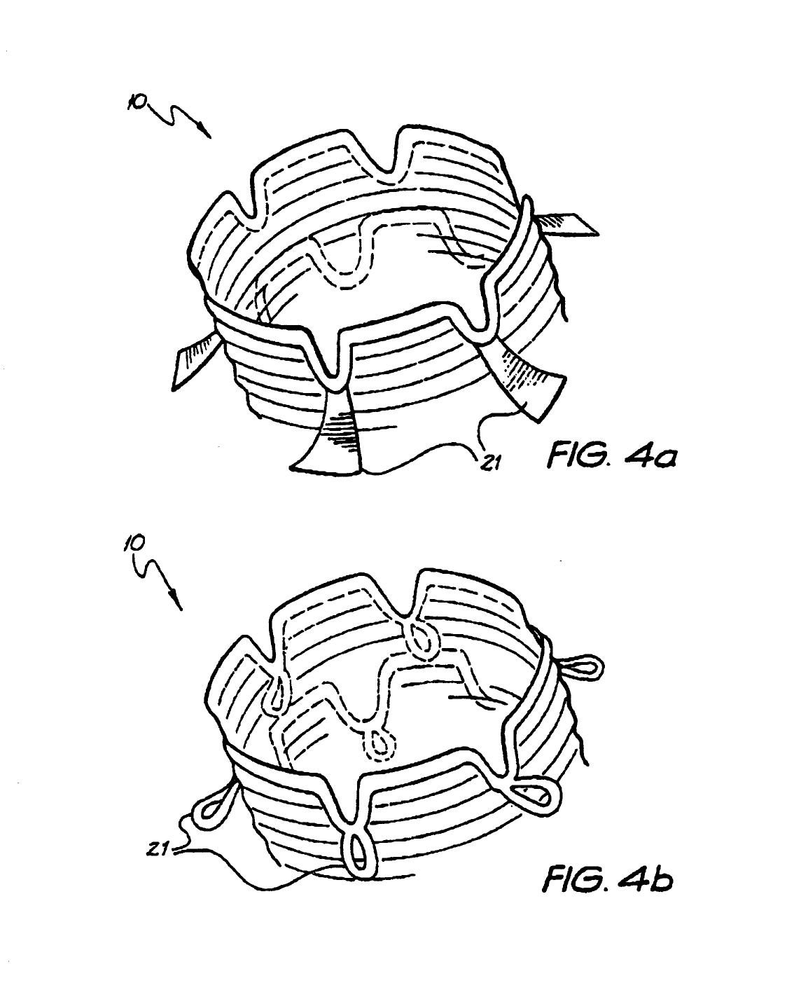

When the device 10 is to be used in the treatment of, for example, an aortic aneurysm, the device 10 is adapted for insertion transfemorally into a patient to achieve bridging and occlusion of the aneurysm present in the aorta 11. As is seen in FIG. 1, the aorta 11 is connected to the left and right femoral arteries 14 and 15. The aortic aneurysm is located between the renal arteries 16 and 17 and the bifurcation of the aorta 18. The device 10 is inserted inside a catheter introduced into one of the femoral arteries 14 and 15 in a leg of the patient in a radially compressed state (FIG. 3a). At this instance, the respective first angular relationships of the engagement members 21 is preferably substantially flat, running along or forming a part of the wall of the device.

Once the catheter is located appropriately with its proximal end in the aorta 11, the device 10 is deployed from the catheter and is caused or allowed to expand into a radially expanded state (FIG. 3b) so that the wall of the device comes into contact with the luminal wall of the aorta 11. As the body of the device 10 expands or is expanded from a radially compressed state (FIG. 3a) to a radially expanded state (FIG. 3b), the engagement members 21 may be caused or allowed to change from their first angular relationship (FIG. 3a) to a second angular relationship (FIG. 3b). In their respective second angular relationships, the engagement members 21 may preferably come into engaging contact with the vessel wall 19. The device then bridges the aneurysm, isolating any thrombosis or gelatinous material associated with the aneurysm outside the device 10 and reducing the risk of embolization. The device is prevented from longitudinal movement within the aorta by virtue of the connection between the engagement members 21 and the vessel wall 19.

When the device 10 is to be used in the treatment of a stenotic lesion, the device 10 is adapted to be inserted into the selected vessel 13 in a patient to achieve radial expansion of the stenosis and patency of the vessel 13. As is seen in FIG. 2, the device is in its radially compressed state (FIG. 3a). It is desirable, for the purpose of introducing the device 10 into the selected vessel 13, for the device to occupy the smallest possible diameter along its length. In this case, the device 10 has the capacity to be introduced percutaneously through a distal (or proximal) and connecting vessel to that in which the device is to be deployed. As above, a catheter may be used to introduce the device 10 into the patient.

Once the device 10 is positioned appropriately, it may be caused or allowed to expand to a radially expanded state (FIG. 3b), and in so doing it may cause the stenotic lesion 12 itself to radially expand. The device 10 may then maintain that position for the remainder of its life, thereby ensuring patency of the vessel. The engagement members 21 will change their position from a first angular relationship to a different second angular relationship and perform their function as described above.

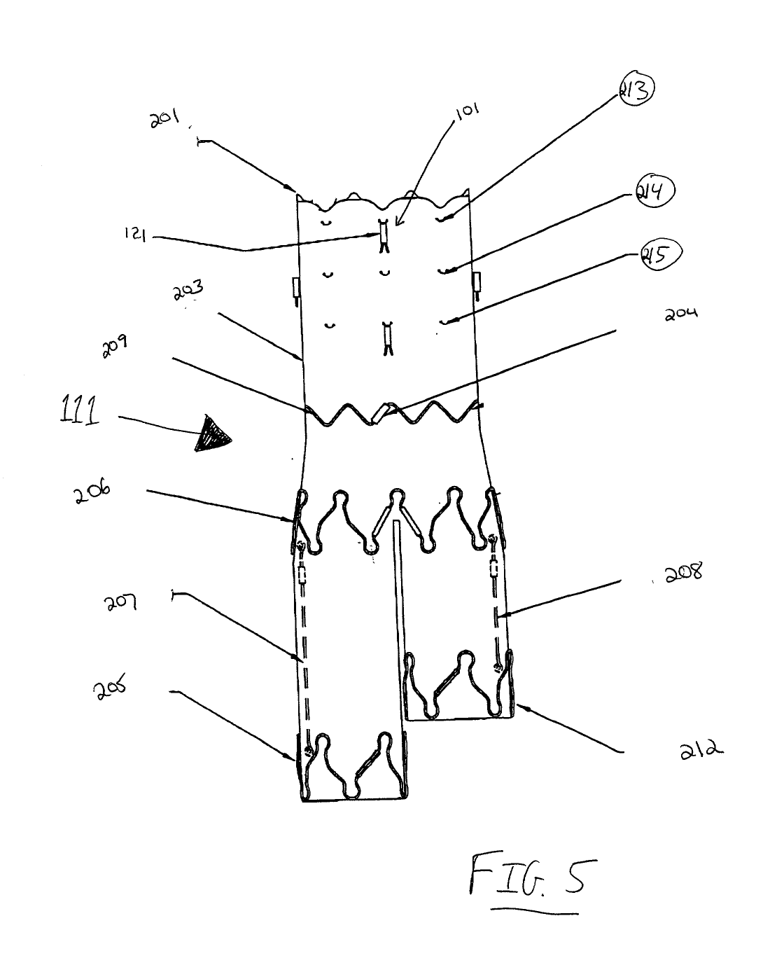

FIG. 5 depicts a preferred embodiment of the device in accordance with the present invention, wherein engagement members are dispersed upon the exterior of a bifurcated prosthesis. As shown in FIG. 5, a bifurcated graft 111 is equipped with crimps 101 dispersed along the exterior wall 203 of the graft 111. This preferred embodiment further possesses several self-expanding wireforms 205, 206, and 212 around the circumference of selected points of the graft 111. There are further included balloon-expandable wireforms 209, 213, 214, and 215. The balloon-expandable wireform 209 is distinguished from balloon-expandable wireforms 213, 214, and 215 by virtue of its being constructed of a circular loop of wire fused together at sleeve 204. Wireforms 213, 214, and 215, however, are constructed of two pieces of wire and said pieces of wire are joined by two crimp sleeves 121. This preferred embodiment further includes longitudinal support wires 207 and 208, which prevent the graft 111 from bucking upon longitudinal deployment from a catheter or similar delivery device.

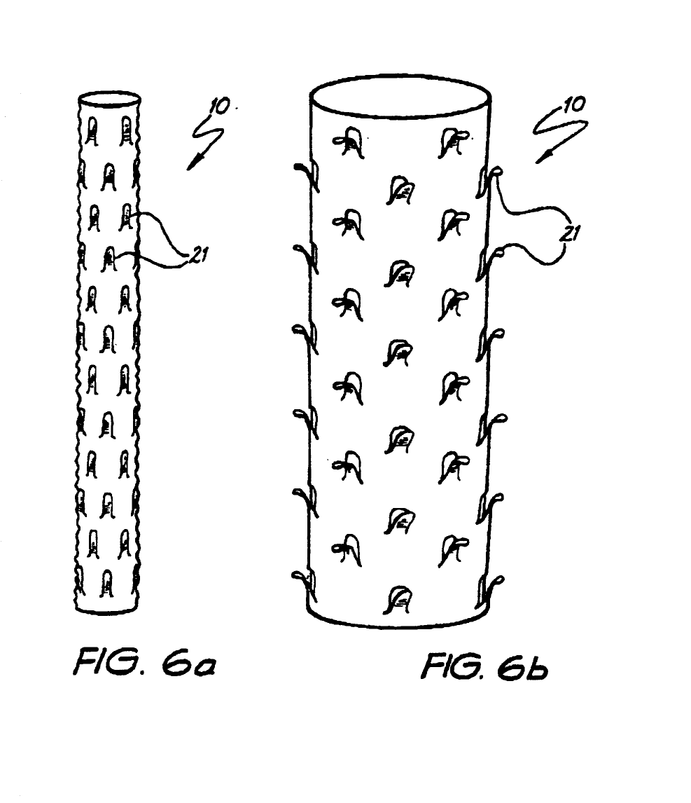

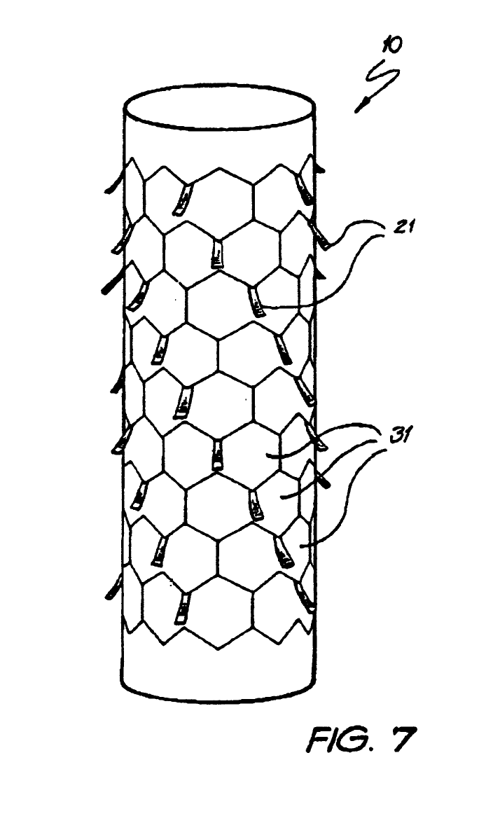

FIGS. 6a and 6b illustrate a further embodiment of the device 10 in its radially compressed and radially expanded states, respectively. In this embodiment, the body of the device 10 is preferably formed of Nitinol, and the engagement members 21 are contiguous with the wall of the device 10, but for a small incision in the wall of the device defining each of the individual engagement members' shapes. Further, in this embodiment, “thermal expansion” is the preferred method for causing the device body 10 to change from a radially compressed state (FIG. 6a) to a radially expanded state (FIG. 6b), and similarly, “heat-aided change” is the preferred method for causing the engagement members 21 to change from a first angular relationship to a second angular relationship. In this embodiment, the device 10 may be manufactured such that the heat pre-treatment of the Nitinol imparts to the engagement members 21 the ability, following a rise in temperature, to splay out from a wall of the device. Thus, although the radially expanded state (FIG. 6b) of the device 10 may be of a fixed diameter, the particular make-up of the components of the wall comprising the engagement members 21, is such that the engagement members 21 may extend further out the from a wall of the device body. An alternative form of this embodiment includes the manufacture of the device wherein the engagement members are surrounded by a substantial area of perforation in a wall of the device 10 (See FIG. 7).

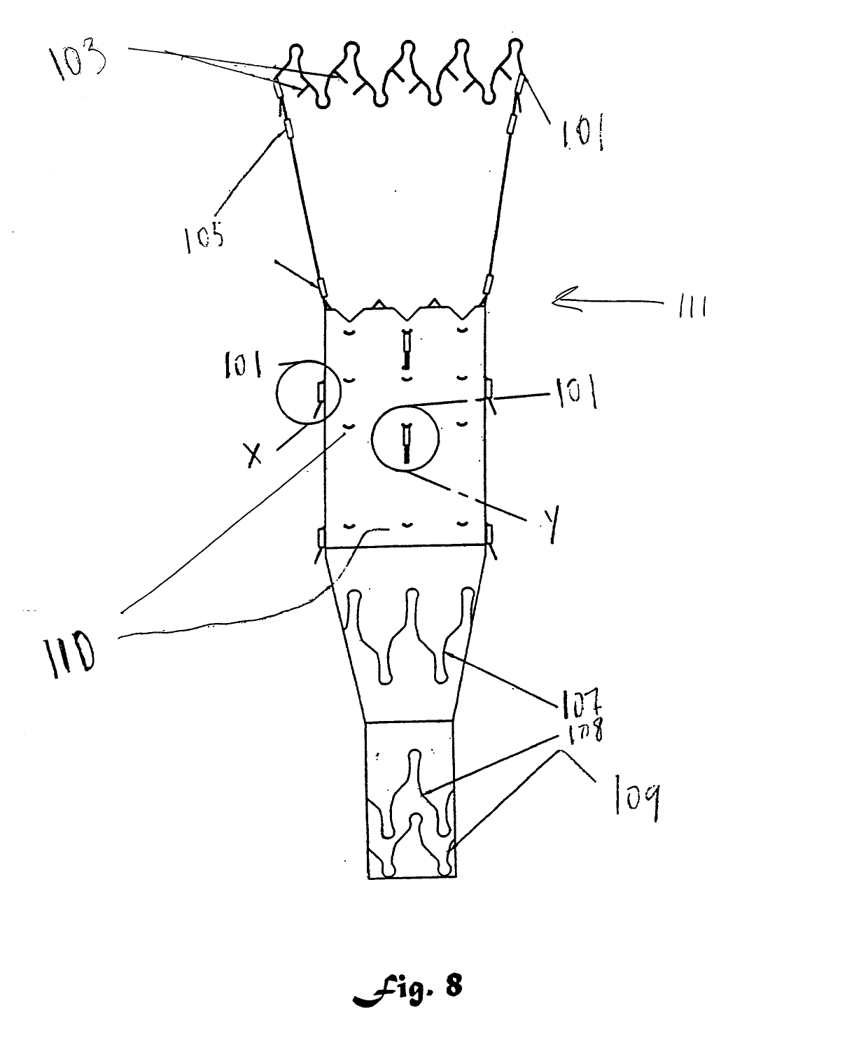

Referring to FIG. 8, the present invention contemplates an enhanced, specifically configured engagement member, or “crimp,” 101, constructed of Elgiloy for example, for securing prosthetic devices within lumens of vessels. The crimps 101 are preferably disposed in a fixed geometric pattern upon an outer surface of an endoluminal graft 111. For example, endoluminal graft 111 in this schematic includes a balloon-expanding wireform 110, attached to two crimps 101, disposed 180° from one another around the circumference of the graft 111. Continuing from proximal to distal ends of endoluminal graft 111, crimps 101 are shown in a preferred pattern above self-expanding wireform stents 107, 108, and 109, interwoven throughout endoluminal graft 111. Thus, the crimps 101 are situated along the outer surface of the endoluminal graft 111 at locations other than solely at the terminal ends of the graft. This feature of the present invention allows the graft to resist migration through a body lumen by applying resistant force along much of the outer surface length of the graft. Moreover, the crimps may be disposed in a configuration upon the outer surface of the device of the present invention in a manner other than a fixed geometric pattern, although this latter geometric pattern is preferred to achieve proper compliance of the device to the wall of the lumen.

Conventional materials may be used for the crimps, such as Nitinol, stainless steel, plastics, tentalum, and Elgiloy, though other malleable, biocompatible materials may be employed as well. In most preferred embodiments, Elgiloy metal wire is used.

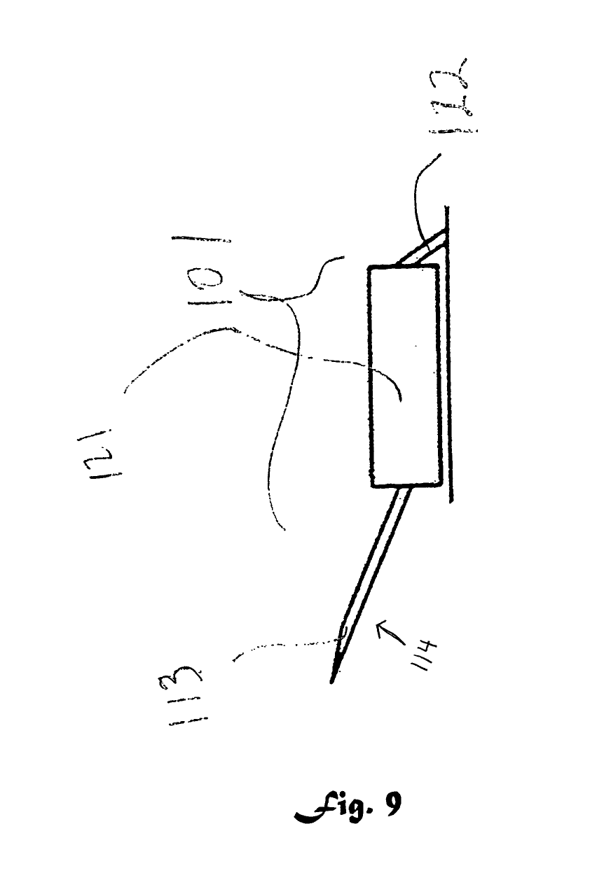

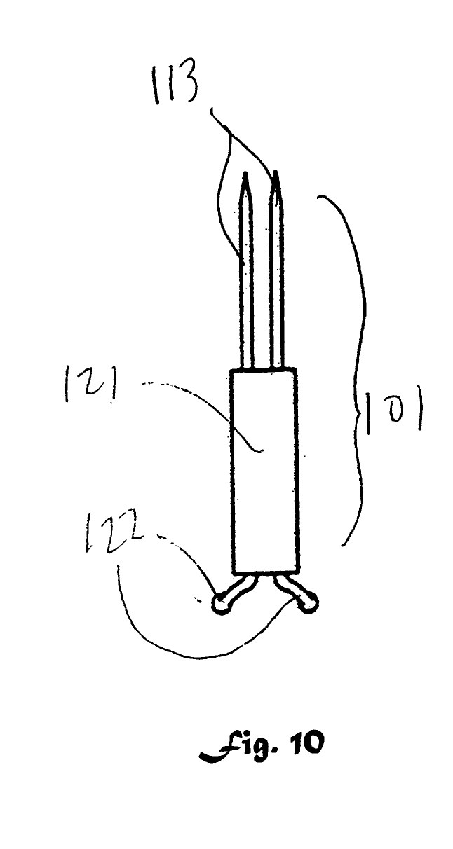

Referring to FIGS. 9 and 10, individual engagement members, or crimps, 101 are preferably composed of a projection extension 114 having at least one generally elongated member 113, a crimp sleeve 121, and at least one positioning attachment member 122. Such crimp sleeve 121, which may also be referred to as a joining member, may be formed by many means known in the art including, but not limited to, crimping, soldering, and/or fusing.

In preferred embodiments, the projection extension 114 has at least two generally elongated members 113, which extend from about 1 mm to about 4 mm from sleeve 121. In most preferred embodiments, two elongated members 113 extend from about 2 mm to about 4 mm from the sleeve 121. Similarly, in most preferred embodiments two positioning attachment members 122 structurally connect sleeve 121 to endoluminal graft 111.

In a preferred embodiment of the present invention, the two positioning attachment members 122 are extensions of undulating filaments extending circumferentially around the body of the graft. These filaments are aligned in an end-to-end configuration to form supportive rings extending primarily along the interior surface of the graft body. As shown in FIG. 5, such filaments may extend through the graft surface in certain embodiments of the invention. See, e.g. wireform 214. As would be realized by one skilled in the art, the interior rings may unitary, that is, formed of only one undulating element. One or more undulations of such element may extend through the surface of the graft, and be configured to be included in an engagement member. In such an embodiment, the filaments may be able to move relative to the device body, upon axial movement or spatial contortion thereof This feature aids in preventing the structure from being damaged by normal movement of the human body in which the device is implanted.

A projection extension angle is defined between the surface of the sleeve 121 (parallel to the surface of the device 111) and the elongated member 113. In preferred embodiments, the projection extension angle is between zero and five degrees. This small projection extension angle enables the involved prosthesis to be adjacent to, but not penetrate, the vessel wall.

In preferred embodiments, sleeve 121 is spatially oriented in such a manner that it is roughly parallel to the outer surface of the endoluminal graft to which it is affixed, as depicted in FIGS. 8 and 9. In most preferred embodiments, this spatial orientation remains the same both before and after deployment of the endoluminal graft. As discussed above, just prior to deployment in a body lumen, a graft is maintained in a manner such that its axial radius is minimized, for ease in surgical insertion of the device. During and after deployment, however, the radius is preferably expanded or allowed to expand within the targeted lumen. The structure of the graft is most preferably composed in such as manner as to allow the sleeve 121 to remain approximately parallel to the outer surface of the graft, even during such radial expansion.

It will be appreciated by persons skilled in the art that numerous variations and/or modifications may be made to the invention as shown in the specific embodiments without departing from the spirit or scope of the present invention as described above. The present embodiments are, therefore, to be considered in all respects as illustrative and not restrictive.

What is claimed is:

1. An endoluminal graft configured to frictionally engage an inner wall of a body vessel comprising:
a graft;
a plurality of engagement members disposed on an outer surface of said graft, wherein the engagement members are configured to frictionally engage an inner wall of a vessel so as to inhibit longitudinal movement of the graft without piercing the vessel wall.

2. The graft of claim 1, wherein said plurality of engagement members are disposed at non-terminal positions of the graft.

3. The graft of claim 1, wherein said plurality of engagement members each comprise:
a projection extension;
a sleeve coupled to said projection extension defining a projection extension angle therebetween; and
a positioning attachment member coupled to said sleeve and positioning the engagement member on the graft.

4. The graft of claim 1, wherein said plurality of engagement members are constructed of a biocompatible, malleable material.

5. The graft of claim 1, wherein said plurality of engagement members are constructed from a material selected from the group consisting of a cobalt-chromium-nickel alloy, a nickel-titanium alloy, stainless steel, plastic, and tentalum.

6. The graft of claim 3, wherein said projection extension angle is from about 0 to about 5 degrees.

7. The graft of claim 3, wherein said projection extension is from about 1 mm to about 4 mm in length.

8. The graft of claim 3, wherein said projection extension is from about 2 mm to about 4 mm in length.

9. The graft of claim 3, wherein said projection extension member comprises first and second generally elongated members.

10. The graft of claim 3, wherein said positioning attachment member comprises first and second members.

11. The graft of claim 3, wherein said sleeve is joined to the positioning attachment member so as to remain substantially parallel to the outer surface of said graft both before and after deployment of said graft in a body vessel.

12. The graft of claim 3, wherein said plurality of engagement members is disposed on the outer surface of said graft in a fixed geometric pattern.

13. An intraluminal device comprising:
a tubular body having first and second ends and an outer longitudinal surface, the tubular body being radially expandable from a radially compressed state to a radially expanded state, and
a plurality of specifically-configured engagement members disposed on an outer surface of the graft, wherein the engagement members are configured to frictionally engage an inner wall of a vessel so as to inhibit longitudinal movement of the graft without piercing the vessel wall.

14. The intraluminal device of claim 13, wherein said plurality of engagement members each comprise:
a projection extension;
a sleeve coupled to the projection extension defining a projection extension angle therebetween; and
a positioning attachment member coupled to the sleeve and positioning the engagement members on the graft.

15. The intraluminal device of claim 13, wherein said plurality of engagement members are constructed of a biocompatible, malleable material.

16. The intraluminal device of claim 13, wherein said plurality of engagement members are constructed from a material selected from the group consisting of a cobalt-chromium-nickel alloy, a nickel-titanium alloy, stainless steel, plastic, and tentalum.

17. The intraluminal device of claim 14, wherein said projection extension is from about 1 mm to about 4 mm in length.

18. The intraluminal device of claim 14, wherein said projection extension is from about 2 mm to about 4 mm in length.

19. The intraluminal device of claim 13, wherein engagement member is disposed on the outer surface of said device in a fixed geometric pattern.

20. The intraluminal device of claim 13, wherein said device is selected from the group consisting of a bifurcated endoluminal prosthesis, an aorto-uniliac stent-graft, and a thoracic aortic pros thesis.

21. An endoluminal graft comprising:
a tubular, biocompatible graft body having a predetermined circumference;
a plurality of undulating filaments extending circumferentially around the graft body and forming a generally ring-shaped configuration, each filament having a first and a second end extending through the graft body and forming an abutting junction between two filaments, wherein the first and second ends of each filament extend through the graft body; and
a joining member securing the junction between the two filaments, wherein the ends of the filaments extend through the joining member and are configured to frictionally engage a wall of a body vessel.

22. The endoluminal graft of claim 21, wherein the graft has a first, radially compressed configuration, and a seconded, expanded configuration.

23. The endoluminal graft of claim 21, wherein said filaments are constructed from a material selected from the group consisting of a cobalt-chromium-nickel alloy, a nickel-titanium alloy, stainless steel, plastic, and tentalum.

24. The endoluminal graft of claim 21, wherein the ends of the filaments extending through the joining member and define a projection extension angle with the joining member.

25. The endoluminal graft of claim 21, wherein the projection extension angle is from about 0 to about 5 degrees.

26. The endoluminal graft of claim 21, wherein the ends of the filaments extending through the joining member extend from about 1 mm to about 4 mm beyond the joining member.

27. An endoluminal graft comprising:
a generally cylindrical, tubular graft body; and
a plurality of rings of circumferentially disposed on an interior surface of the graft body, each of the rings comprising:
a first wireform having a first end and a second end, the first and second ends each projecting through the graft body to an exterior surface thereof;
a second wireform having a first end and a second end, the first and second ends each projecting through the graft body to the exterior surface thereof; and
joining members positioned adjacent to the exterior surface of the endoluminal graft, a first selected joining member joining the first end of the first wireform to the first end of the second wireform, and a second joining member joining the second end of the first wireform to the second end of the second wireform, wherein a portion of each of the wireforms extends through the joining member and defines a projection extension.

28. The endoluminal graft of claim 27, wherein said projection extensions are configured so as to frictionally engage an inner wall of a body vessel.

29. An endoluminal graft comprising:
a pliable, tubular graft body having an interior surface and an exterior surface; and
a plurality of generally circular wireforms circumferentially disposed along the interior surface of said graft body, said wireforms each being composed of at least two undulating wires, a first wireform being joined to a second wireform to form a joined pair of wire ends, each pair of wire ends extending through the graft body to the exterior surface thereof such that there is relative movement between the graft body and the wire ends;
wherein the wire ends define a projection extension, the projection extension configured to frictionally engage a wall of a body vessel.

30. The endoluminal graft of claim 29, wherein said rings are disposed adjacent to the interior surface of the graft body.

31. The endoluminal graft of claim 29, wherein said pliable, tubular graft body is expandable from a first radially compressed configuration to a second expanded configuration;
wherein the wire ends are joined together by means of a sleeve, a portion of said wire ends extending beyond said sleeve so as to define a projection extension; and
further wherein said sleeve remains substantially parallel to said graft before, during and following deployment.

32. A method of preventing migration of an endoluminal graft through a body lumen comprising the steps of:
providing an endoluminal graft having a plurality of engagement members disposed on an outer surface of the graft, wherein the engagement members are configured to frictionally engage an inner wall of a vessel so as to inhibit longitudinal movement of the graft without piercing the vessel wall; and
surgically disposing the endoluminal graft in a body lumen such that the engagement members frictionally engage the inner wall of the vessel without piercing the vessel wall.
